(19) United States
(12) Patent Application Publication
(10) Pub. No.: US 2021/0106309 A1
(43) Pub. Date: Apr. 15, 2021

(54) METHOD FOR ULTRASONIC DIAGNOSTIC IMAGING PROCEDURES

(71) Applicant: KONINKLIJKE PHILIPS N.V., EINDHOVEN (NL)

(72) Inventors: Robert Mesaros, Bothell, WA (US); Shruti Pai, Seattle, WA (US)

(21) Appl. No.: 16/500,122

(22) PCT Filed: Apr. 3, 2018
(86) PCT No.: PCT/EP2018/058417

§ 371 Date: Oct. 2, 2019

Related U.S. Application Data
(60) Provisional application No. 62/481,014, filed on Apr. 3, 2017.

(51) Int. Cl. A61B 8/00; A61B 90/00

(52) U.S. Cl. CPC A61B 8/4218; A61B 90/37; A61B 8/40; A61B 2090/378; A61B 8/467; A61B 8/4405; A61B 8/461

(57) ABSTRACT

An ultrasonic diagnostic imaging system (10) and method are described for conduct of an ultrasonic image-guided invasive procedure. An ultrasound system having a touchscreen display (30) located at the distal end of an articulating display arm (32) is positioned on one side of a patient table (110). The clinician performing the procedure is located on the other side of the patient table. The touchscreen display is extended over the patient table by articulation of the articulating arm. The position of the touchscreen display is adjusted by the clinician for optimal viewing in front of the clinician and above the patient table.

This invention relates to medical diagnostic ultrasound systems and procedures, in particular, to ultrasound systems for use in image-guided procedures.

One of the advantages of ultrasound imaging, in addition to its use of non-ionizing radiation, is its portability. Even the largest and most complex ultrasound systems, such as the system shown in U.S. Pat. No. 6,516,215 (Roundhill), are wheeled, enabling them to be brought to a patient's bedside for a diagnostic procedure. Typically, the sonographer rolls the ultrasound system to the side of the patient's bed and positions himself or herself at the side of the bed in front of the ultrasound system display and keyboard. The sonographer holds the ultrasound probe against the body of the patient with one hand while using the other to manipulate the controls of the ultrasound system in order to obtain the best desired images. The sonographer concentrates on the images on the display screen, looking for the right view of the patient's anatomy and image clarity. Experienced sonographers rarely have to look at the probe as it is manipulated, as the ultrasound images on the display screen provide all the visual clues needed to position and reposition the probe against the patient. The imaging exam proceeds with the sonographer devoting virtually full attention to the images on the display screen.

Such is not the case, however, when a clinician is performing an image-guided procedure such as a needle insertion. In such procedures, the clinician must not only observe the ultrasound image and often hold the ultrasound probe, but must also devote focused attention on the insertion and guidance of the needle or other invasive device. In the case where the probe is hand-held rather than taped or strapped to the patient, the procedure requires considerable skill and dexterity. When the ultrasound system is alongside the patient table or bed as in the standard imaging exam described above, the clinician must frequently turn his or her head to observe the display screen on the ultrasound system, noting the position of the instrument inside the body and then turning back to the patient, all while manipulating and guiding the invasive instrument. Thus, it would be desirable to be able to perform an ultrasound image-guided procedure with the display screen positioned as close to the site of the procedure as feasible, so that the clinician can quickly avert his or her eyes to the display screen and back to the site of the instrument procedure without turning the head or body. It would further be desirable to have the ultrasound system controls equally accessible to make any system adjustments necessary to optimize the guiding image on the display screen.

In accordance with the principles of the present invention, an ultrasound system and method are described which facilitates an image-guided invasive procedure. The ultrasound system comprises a display system with touchscreen controls which is mounted on a thin, lightweight wheeled stand similar to a medical IV pole. The touchscreen display is located at the distal end of an adjustable articulating arm that is mounted on the stand. The ultrasound system is positioned at the side of the patient table or bed, opposite the side where the clinician performing the procedure is located. The articulating arm is extended across the patient table toward the position of the clinician during the procedure. The clinician can manipulate the touchscreen display and its articulating arm so that the touchscreen is positioned above the patient and the instrument insertion site. With the display screen positioned in this way, the system is out of the way of the clinician and the screen is located so that the internal anatomy and path of the invasive device can be seen without movement of the clinician other than averting the clinician's eyes. The touchscreen controls of the ultrasound system are also immediately in front of the clinician for easy access without diverting attention from the site of the insertion procedure.

In the drawings:

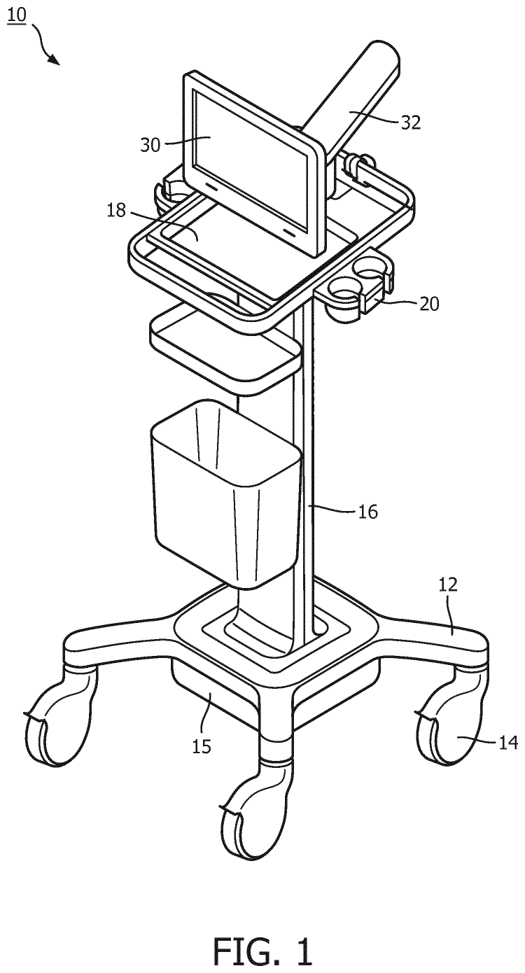

FIG. 1 is a perspective view of an ultrasound system of the present invention.

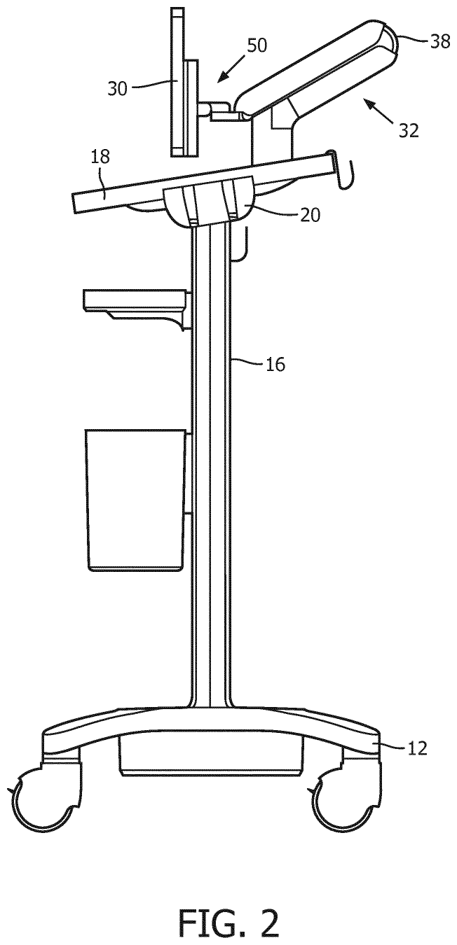

FIG. 2 is a side view of the ultrasound system of FIG. 1.

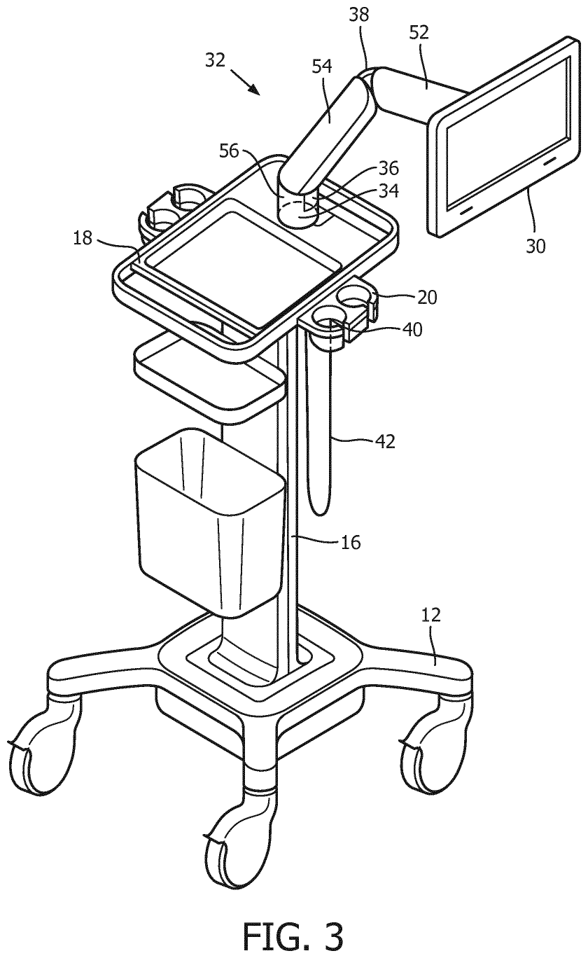

FIG. 3 is a perspective view of the ultrasound system of FIG. 1 with the articulating arm for the touchscreen display in an extended position.

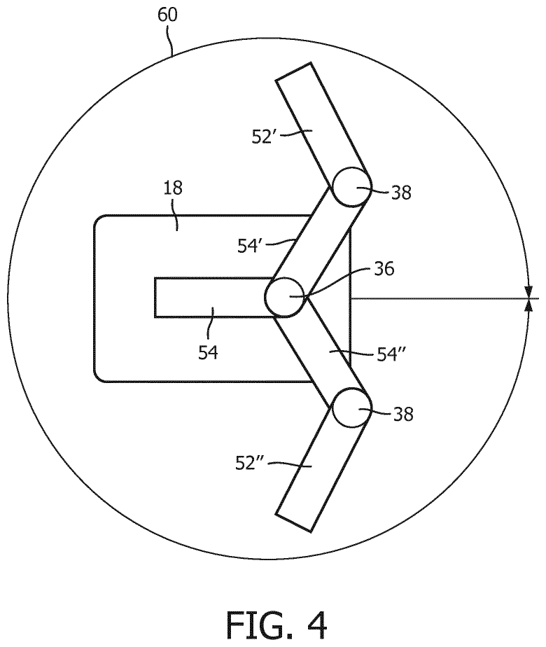

FIG. 4 illustrates the range of articulation of the articulating arm of the ultrasound system of FIGS. 1-3.

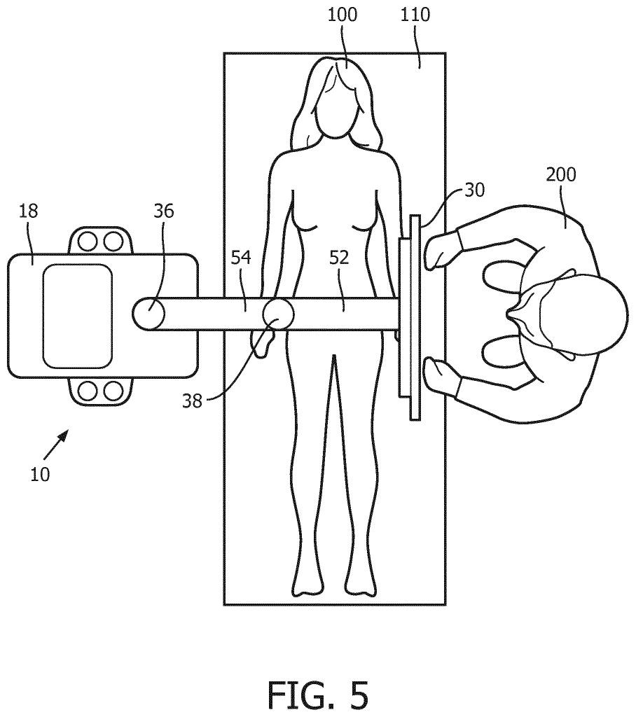

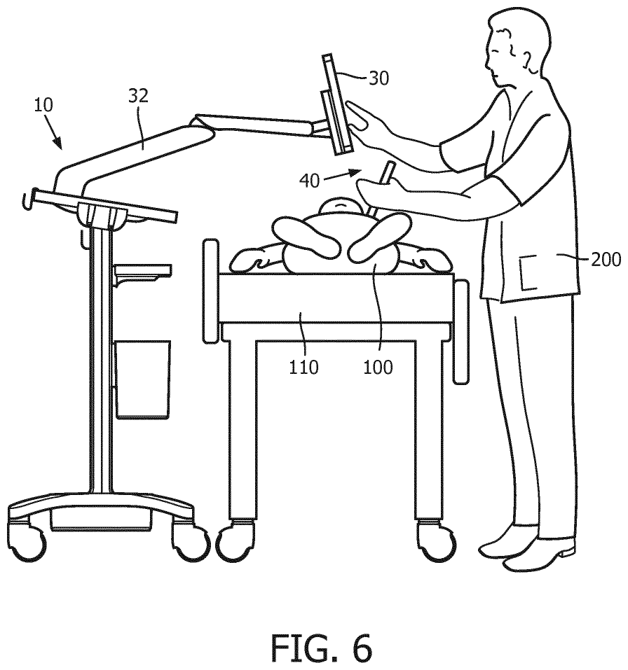

FIGS. 5 and 6 illustrate the ultrasound system of FIGS. 1-4 being used for an image-guided procedure in accordance with the present invention.

Referring first to FIGS. 1 and 2, an ultrasound system 10 of the present invention is shown in a perspective view. The system 10 comprises a base 12 with lockable wheels 14 for moving the system to the location where an image-guided procedure is to be performed such as a surgical suite. Located in the base 12 is a power supply module 15 which supplies power for the system. Extending upward from the base 12 is a central pole 16. A tray 18 is located at the top of the pole which can hold instruments to be used in a procedure. Other accessories such as an additional tray and a basked may be mounted on the pole as shown in the drawings. Probe holders 20 are located on the sides of the tray to hold ultrasound probes which are coupled to the system and not currently being used for imaging. FIG. 3 illustrates an ultrasound probe 40 held in one of the probe holders. The cable 42 of probe 40 is plugged into a probe jack located on the underside of tray 18. A touchscreen display 30 is mounted on top of the tray by an articulating arm 32, which enables the touchscreen 30 to be manipulated and positioned for an image-guided procedure as discussed below. Power for the touchscreen display 30 is provided by wiring (not shown) extending through the articulating arm 32 and pole 16 to the power supply module 15. Wiring also extends through the articulating arm from the probe jack to the touchscreen display.

In FIG. 2 the articulating display arm 32 is shown in its stowed position. The articulating arm is locked in this position when the system is moved for safety purposes. Further details of an articulating arm with this capability are described in US pat. appl. pub. no. 2008/0234577 (Murkowski et al.) When the articulating arm 32 is unlocked so it can be articulated from its stowed position as shown in FIG. 3, the lower arm section 56 can be pivoted around a vertical axis extending through it base 34. A second articulation joint 36 connects the lower arm section 56 and the middle arm section 54, which permits the middle arm section 54 to be raised and lowered. The middle arm section 54 is connected to an upper arm section 52 by an articulation joint 38 which permits the upper arm section to be raised and lowered and pivoted about a vertical axis with respect to the middle arm section. The upper arm section 52 is coupled to the back of the touchscreen display by another articulation joint 50 at the distal end of the articulating arm, shown in FIG. 2, which allows the display 30 to be tilted upward and downward and to be pivoted about a vertical axis with respect to a user.

The articulating arm 32 has a wide range of motion so that the touchscreen display 30 can be positioned in a convenient location for a procedure as illustrated diagrammatically in FIG. 3. This top view of the articulating arm 32 and tray 18 illustrate that the middle arm section 54, in addition to being capable of being raised and lowered, can also pivot a full 360° around the center axis of articulation joint 36, as indicated by the circular range-of-motion arrow 60. This illustration shown the middle arm section 54 in three positions 54, 54′, and 54″. The upper articulation joint 38 provides additional range of motion as it enables the upper arm section 52 to be pivoted about the joint as illustrated by positions 52′ and 52″. The upper articulation joint also permits the upper arm section to be raised and lowered with respect to the middle arm section.

This wide and versatile range of motion of the articulating arm 32 enables the ultrasound system 10 to be positioned in a new way for a diagnostic or image-guided procedure as illustrated in FIGS. 5 and 6. FIG. 5 is a top view of ultrasound system 10 being used for an image-guided procedure such as a biopsy or anesthesiology procedure, and FIG. 6 is a side view. A patient 100 is in a prone position on a surgical bed or table 110 and the clinician 200 who is performing the procedure is located on the right side of the table 110. Instead of positioning the ultrasound system on the same side of the patient table 110 as the position of the clinician as is conventionally done, the system 10 is positioned on the left side of the table as shown in the drawing. When the ultrasound system is secured in this position by locking the wheels, the articulating arm can be swung around and articulated to an extended position over the patient 100 and toward the position of the clinician 200 as shown in the drawing. The arm 32 swings around its vertical pivot axis at the base 34, then is articulated at articulation joints 36 and 38 so that articulating arm sections 54 and 52 extend over the patient on the surgical table. The clinician 200 can then grab the touchscreen display 30 and position the display where desired above the table 110 and can further tilt the touchscreen display at an optimal viewing angle by virtue of articulation joint 50. With the touchscreen display positioned in front of the clinician and above the patient 100, the clinician can use both hands to hold the ultrasound probe and any instruments needed for the procedure without further interaction with the display. Since the touchscreen display also has control touch points on the screen for control of the ultrasound system, the clinician can, if needed, adjust these system touchscreen controls manually to optimize the system for best visualization of the procedure.

A preferred implementation of the probe 40 for an ultrasound system of the present invention will comprise all of the circuitry and software necessary to send and receive ultrasound signals and process the signals into an ultrasound image for display on the touchscreen display. Such probes are available from Philips Healthcare of Andover, Mass., and are also used with Philips' Visicu ultrasound system.

It should be noted that an ultrasound system suitable for use in an implementation of the present invention, and in particular the component structure of the ultrasound system of FIGS. 1-5, may be implemented in hardware, software or a combination thereof. The various embodiments and/or components of an ultrasound system, for example, the touchscreen display and the ultrasound probe described above, also may be implemented as part of or using one or more computers or microprocessors. The computer or processor may include a computing device, an input device, a display unit and an interface, for example, for accessing the Internet. The computer or processor may include a microprocessor. The microprocessor may be connected to a communication bus, for example, to access a PACS system or the data network for importing training images. The computer or processor may also include a memory. The memory devices may include Random Access Memory (RAM) and Read Only Memory (ROM). The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” or “processor” or “workstation” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of these terms.

The computer or processor executes a set of instructions that are stored in one or more storage elements, in order to process input data. The storage elements may also store data or other information as desired or needed. The storage element may be in the form of an information source or a physical memory element within a processing machine.

The set of instructions of an ultrasound system including those controlling the acquisition and processing of ultrasound images as described above may include various commands that instruct a computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules such as ones executing the simulation and processing of the equations of the reverberation signal processor described above. The software also may include modular programming in the form of object-oriented programming. The processing of input data by the processing machine may be in response to operator commands, or in response to results of previous processing, or in response to a request made by another processing machine.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

1. A method for conducting an ultrasonic image-guided procedure using an ultrasound system with an ultrasound image display mounted on a distal end of an articulating arm, comprising:
positioning the ultrasound system on a first side of a patient table;
extending the articulating arm over the patient table toward a clinician position on a second side of the patient table; and
positioning the ultrasound image display for viewing from the clinician position,
wherein extending the articulating arm comprises pivoting middle and upper articulating arm segments about vertical axes of first and second articulation joints respectively, and further comprises raising or lowering the middle and upper articulating arm segments via said first and second articulation joints respectively.

2. (canceled)

3. (canceled)

4. The method of claim 1, wherein extending the articulating arm further comprises pivoting a lower articulating arm segment about a vertical axis.

5. (canceled)

6. (canceled)

7. The method of claim 1, wherein positioning the ultrasound image display further comprises raising or lowering the ultrasound image display with respect to the patient table.

8. The method of claim 7, wherein positioning the ultrasound image display further comprises tilting the ultrasound image display upward or downward.

9. The method of claim 8, wherein positioning the ultrasound image display further comprises adjusting the position of the ultrasound image display with respect to the patient table from the clinician position.

10. The method of claim 1, wherein the ultrasound image display further comprises a touchscreen display, and further comprising:
controlling the operation of the ultrasound system using the touchscreen display.

11. The method of claim 10, wherein controlling the operation of the ultrasound system further comprises touching touch points on the touchscreen display.

12. The method of claim 11, wherein controlling the operation of the ultrasound system further comprises touching touch points on the touchscreen display from the clinician position.

13. A system for conducting an ultrasonic image-guided procedure, the system comprising
an ultrasound cart;
an ultrasound image display; and
an articulating arm coupling the cart to the image display, the articulating arm comprises a middle arm segment and an upper arm segments configured to:
pivot about vertical axes of first and second articulation joints respectively, and
move upward or lower relative to the ground about the first and second articulation joints.
